(19) United States
(12) Patent Application Publication
(10) Pub. No.: US 2023/0409742 A1
(43) Pub. Date: Dec. 21, 2023

(54) NETWORK INDEPENDENT MEDICAL FORM APPLICATION FOR GENERATING MEDICAL FORMS UTILIZING COMMON CONTENT STORAGE AND PATIENT EMR FROM PERMITTED EMR DATABASES

(71) Applicant: Cortisoul Innovations, LLC, London, KY (US)

(72) Inventors: Syed Kamran Hasni, London, KY (US); Mary Christina Perkins, Lily, KY (US); Anita Maria Swafford, Bryant's Store, KY (US)

(21) Appl. No.: 18/239,680

(22) Filed: Aug. 29, 2023

Related U.S. Application Data
(63) Continuation-in-part of application No. 16/953,800, filed on Nov. 20, 2020.
Application No. 16/953,800 is a continuation-in-part of application No. 15/530,107, filed on Dec. 5, 2016, now abandoned.
(60) Provisional application No. 62/339,367, filed on May 20, 2016.

(51) Int. Cl. G06F 21/62; G16H 10/60; G06F 40/40; G06F 3/16

(52) U.S. Cl. CPC G06F 21/6245; G16H 10/60; G06F 40/40; G06F 3/165

(57) ABSTRACT

Implementations set forth herein relate to a medical consent application and/or system that can securely retrieve EMR data in a variety of formats and generate consent forms that can be specific to a particular medical process, thereby creating a more universal consent process across different medical provider networks. Any executed consent form can then be converted back to an original format of a source EMR database, thereby allowing medical providers and/or other affiliated entities to securely access consent data via their own EMR database. Customized explanatory content can be stored by, or otherwise accessible to, the medical consent application, thereby allowing patients to view explanatory content during consent processes. A record of what a patient viewed during a consent process can thereafter be stored in consent resource data that is generated for, and sent back to, the EMR database.

### CROSS REFERENCE TO RELATED APPLICATIONS

This application is a continuation-in-part application of application Ser. No. 16/953,800 filed Nov. 20, 2020, which is incorporated herein by reference, along with any other application from which application Ser. No. 16/953,800 claims priority; application Ser. No. 16/953,800 is a continuation-in-part of application Ser. No. 15/530,107 filed Dec. 5, 2016, which is also incorporated herein by reference, along with any other application from which application Ser. No. 15/530,107 claims priority; application Ser. No. 15/530,107 is a Non-Provisional application that claims priority to Provisional Application No. 62/339,367 filed May 20, 2016, which is also incorporated herein by reference, along with any other application from which Provisional Application No. 62/339,367 claims priority.

### BACKGROUND

Promulgation of the ACA (Affordable Care Act) pushed many medical provider networks to become more universal in many of the ways they operate and share data. This push resulted in the discovery of several technical issues previously unbeknownst to providers, patients, and other medical staff. One issue is that healthcare systems across the globe cause fragmentation of healthcare information and often fail to provide any interoperability between healthcare networks. As an example, a medical application that operates in a particular provider network would not be operable in other networks because of the differences in authentication and/or protocols for securing electronic medical records (EMR) databases, and/or because some networks still operate using paper records. In other words, a provider that utilizes a particular medical application for a particular provider network would have no way of interfacing with any other medical networks-much less in anyway that is organized, efficient, and standardized. For example, a medical application utilized by a region of providers may be able to access their network's EMR data, but the same medical application would not be useful in any other medical network. As a result, medical providers across different regions are required to utilize different medical applications that store resources, such as forms and templates, according to different protocols and/or formats. For example, forms and templates are stored duplicatively across numerous medical network servers and devices, thereby unnecessarily wasting memory and other resources of those servers and devices.

This issue can be particularly apparent during patient intake and discharge, during which certain medical records are modified and/or communicated to other associated medical entities. For instance, before a patient undergoes a medical procedure, medical personnel describe the procedure to the patient, including any side effects or risks to the procedure, and then obtain the patient's consent to perform the operation. Typically, this is done in person, with a doctor or nurse describing the procedure and answering the patient's questions, which may not be recorded or otherwise embodied in any standardized format for communicating between providers and/or networks.

Certain technical issues can be exacerbated by differences in consent and discharge data and procedures among hospital networks, which may manage data (e.g., executed consent documents, discharge documents, etc.) in different formats and/or inconsistently make certain data available to patients and/or providers. For example, contextual data for an executed consent or discharge document may not be available because of limitations of the systems regularly relied upon by providers and medical personnel. Therefore, any additional noteworthy information apparent during patient intake and/or discharge would not be stored in a way that would amend EMRs that future providers might utilize to define a scope of future treatment, send reminders, and/or otherwise manage patient communications.

In some instances, as a result of deficiencies in interoperability of healthcare systems and/or a lack of electronic procedures for obtaining consent, certain consent procedures may be unnecessarily duplicated for a given patient. As a result, technical resources such as the memory of any devices storing duplicative scanned forms, network bandwidth consumed sharing duplicative consent data, and/or any other medical device/network resources would be wasted. Furthermore, and as a result, any contextual data or content (e.g., explanation videos, audio, etc.) that serves as the basis for what a patient is consenting to may not be reliably available after the consent and/or discharge procedure because of deficiencies in EMR technology and/or a lack of reliance on any substantive information retained during consent procedures. Therefore, a patient or family member that needs to receive reminders or access a patient's electronic account to refresh their recollection of, or even confirm, any data received by a patient before, and/or after, a procedure may receive inconsistent or incomplete information from a hospital or provider.

Moreover, preserving privacy of a patient's medical records and/or other health data can also prove difficult in the face of existing technical deficiencies of existing medical applications. For example, although third parties may be able to develop explanation videos, text-to-speech technologies, and other advancements to explain a procedure or a post-procedure therapy to a patient, autonomously presenting such content to a patient during intake or discharge could require accessing data from a medical provider or network-without compromising a patient's privacy.

Having all these factors in mind, there is clearly a need for a system, device, or application that: (1) can reduce the amount of duplicatively consumed data storage across medical networks and systems, (2) recognizes the limitations of provider and patient resources, (3) is interactive across provider networks, personal devices, and servers, and (4) preserves a patient's privacy while simultaneously allowing them to learn about their upcoming medical procedure, and to receive informative reminders.

### SUMMARY

Implementations set forth herein relate to systems, methods, and apparatuses that provide a medical application that reduces the amount of duplicative data across medical networks while also improving interoperability of applications across different medical provider networks and abiding by security and privacy standards implemented by various provider networks. For example, instances of the medical application (i.e., medical consent application) operating at different client devices of different medical provider networks can access a common content storage for generating forms (e.g., treatment consent forms, discharge forms, appointment forms, etc.), which the medical application can append with patient data from a respective medical network's own EMR database. When the medical application is adopted globally for various different medical networks, the savings of memory by using the common content storage is significant. Furthermore, server processing bandwidth and energy consumption would also be preserved globally. The medical application further accomplishes this by implementing a login protocol that can authenticate each provider in their respective network with a respective instance of the medical application, and also establishing a secure connection between the medical application and that respective network's EMR database. This latter connection can be secured using an appropriate key value for establishing a token session with that respective network EMIR database, and that key value can be stored with a variety of different key values for various other medical networks. These and other implementations are further detailed herein, along with their technical benefits and achievements.

It should be appreciated that all combinations of any concepts described herein are contemplated as being part of the subject matter disclosed herein. For example, all combinations of claimed subject matter appearing towards the end this disclosure are contemplated as being part of the subject matter disclosed herein.

In some implementations, a method implemented by one or more processors is set forth as including operations such as receiving, at a medical consent application of a client computing device, user credentials for providing a user with access to provider-specific functionality of the medical consent application, wherein the medical consent application provides functionality for multiple different medical providers. The method can further include determining, based on the user credentials, a key value and a provider-specific identifier that are stored in association with the user credentials by the medical consent application or another device associated with the medical consent application. The method can further include establishing, using the key value and the provider-specific identifier, a secure connection between the medical consent application and the separate computing device that provides access to a medical records database, wherein establishing the secure connection includes providing the key value and the provider-specific identifier to the separate computing device to cause the separate computing device to share a token value with the medical consent application. The method can further include determining, by the medical consent application or another application, a format of a plurality of formats that the medical records database stores provider patient data for a patient of the user. The method can further include generating, based on the format for the medical records database, patient resource schema data using the provider patient data, wherein the provider patient data is accessed during a token session between the medical consent application and the separate computing device, and wherein the patient resource schema data: is generated in a separate format from the format of the provider patient data, and includes a field with a value for an encrypted reference to the patient. The method can further include determining, based on the provider patient data, that the patient is scheduled to undergo a medical procedure that can require consent from the patient, or an authorized representative of the patient, before the medical procedure is initiated by a provider or the user. The method can further include accessing, by the medical consent application and based on the medical procedure, procedure schema data that is stored in the separate format, wherein various medical procedure schema data is accessible to the medical consent application to generate medical consent forms for multiple different medical procedures for the multiple different medical providers. The method can further include generating, by the medical consent application and based on the procedure schema data, consent resource schema data to be generated in response to determining that the patient is scheduled to undergo the medical procedure that can require the consent, wherein the consent resource schema data: is generated in the separate format, identifies the medical procedure, and includes the encrypted reference to the patient or a different encrypted reference to the patient. The method can further include causing, by the medical consent application, a patient consent form to be rendered at a display interface of the client computing device using the consent resource schema data, wherein the patient consent form includes natural language content embodied in fields of the procedure schema data and that describes the medical procedure. The method can further include determining that the patient, or the authorized representative of the patient, signed the patient consent form using the medical consent application or a separate application, wherein the patient consent form is signed via direct input to the display interface of the client computing device or other client device. The method can further include generating, by the medical consent application, updated consent resource schema data in response to determining that the patient consent form was signed, wherein the updated consent resource schema data includes another encrypted reference to the patient and indicates the patient consent form was signed. The method can further include converting the updated consent resource schema data into formatted consent resource data, wherein converting the updated consent resource schema data includes changing the separate format of the updated consent resource data to the format of the medical records database. The method can further include providing, by the medical consent application, the formatted consent resource data to the separate computing device during the token session or a subsequent token session between the client computing device and the separate computing device.

In some implementations, the method can further include determining that the user or a medical services provider has caused a particular instance of procedure resource data to be generated, wherein determining that the patient is scheduled to undergo the medical procedure that can require consent from the patient, or the authorized representative of the patient, is performed in response to determining that the medical services provider has caused the particular instance of procedure resource data to be generated. In some implementations, the provider patient data is stored in the medical records database as one or more PDF, TIFF, JPEG, PNG, and/or BMP files, and the patient resource schema data is stored separately from the medical records database in a JSON, SQL, and/or XML format.

In some implementations, generating the consent resource data includes: causing the consent resource schema data to include a consent field with a first consent value that indicates the patient has not consented to the medical procedure, wherein a second consent value is provided in another consent field in the updated consent resource schema data and the second consent value provides an indication that the patient, or the authorized representative of the patient, consented to the medical procedure. In some implementations, generating the consent resource schema data includes: causing the consent resource data to include a media field with a media value that provides a link to, and/or a description of, medical procedure information to be presented to the patient, or the authorized representative of the patient, via the interface of the client computing device.

In some implementations, generating the consent resource data includes: causing the consent resource schema data to include a media acknowledgement field with a first acknowledgement value that indicates the medical procedure information has not been accessed, wherein a second acknowledgement value is provided in another media acknowledgement field in the updated consent resource schema data, and the second acknowledgement value indicates the medical procedure information was accessed. In some implementations, the second acknowledgment value is embodied in the formatted consent resource data that is provided to the separate computing device.

In other implementations, a method implemented by one or more processors is set forth as including operations such as determining, by a medical consent application that is accessible via a client computing device, that a patient is to be presented with a consent form for consenting to a medical procedure, wherein the patient is identified in patient resource data that is stored in a medical provider computing device that is separate from the client computing device. The method can further include causing, in response to determining that the patient is to be presented with the consent form, consent resource data to be generated, wherein the consent resource data identifies the medical procedure and includes an encrypted reference to the patient resource data that identifies the patient. The method can further include determining that the patient, or an authorized representative of the patient, provided express consent for the medical procedure at the consent form, wherein the express consent is received from the patient, or the authorized representative of the patient, via direct user input to the client computing device that provides access to the consent form. The method can further include causing, by the medical consent application, updated consent resource data to be generated and stored at the medical provider computing device in response determining that the express consent for the medical procedure as received, wherein the updated consent resource data includes an indication that the patient, or the authorized representative of the patient, consented to receiving the medical procedure.

In some implementations, determining that the patient is to be presented with the consent form for consenting to the medical procedure includes: determining that the medical records database includes medical procedure resource data, wherein the medical procedure resource data includes another encrypted reference to the patient resource data and identifies the medical procedure. In some implementations, causing the consent resource data to be generated includes: determining a location of an image file and/or a video file that provides supplemental information describing the medical procedure, and causing the consent resource data to be generated with a link or an address for the image file and/or the video file, wherein the image file and/or the video file are rendered at a graphical user interface (GUI) of the client computing device during or before the patient, or the authorized representative of the patient, providing the express consent at the consent form.

In some implementations, the method can further include determining that the patient received the medical procedure subsequent to the express consent being provided at the consent form; and causing, based on determining that the user received the portion of the medical procedure, discharge consent resource data to be generated, wherein the discharge consent resource data identifies the medical procedure, and is accessible to the medical consent application, and includes another encrypted reference to the patient resource data stored at the medical records database. In some implementations, the method can further include determining that the patient received the medical procedure subsequent to express consent being provided at the consent form; and causing, based on determining that the patient received the medical procedure, appointment resource data to be generated, wherein the appointment resource data: includes a reference to a file embodying post-procedure information for the medical procedure, and specifies a time that a reminder will be communicated to a phone number and/or an email address of the patient or the authorized representative of the patient.

In some implementations, the method can further include determining that the patient, or the authorized representative of the patient, provided an indication that a separate medical provider of the patient does not have an account stored at the medical provider computing device or is not associated with a network of the medical provider computing device; and causing, by the medical consent application, the updated consent resource data to be converted to a separate format and communicated to an electronic address of the separate medical provider. In some implementations, the separate format of the updated consent resource data includes a consolidated clinical document architecture (CCDA) format.

In yet other implementations, a method implemented by one or more processors is set forth as including operations such as causing a medical consent application to generate patient resource data using provider patient data that is stored in a medical records database, wherein the provider patient data is stored in a first format in the medical records database, and the patient resource data is generated in a second format. The method can further include determining that a patient is to undergo a medical process that can require consent from the patient, or an authorized representative of the patient, before the medical process is initiated by a provider, wherein the patient is identified in the provider patient data that is stored in the medical records database, and the patient resource data includes an encrypted reference to the patient. The method can further include causing consent resource data to be generated in response to determining that the patient is scheduled to undergo the medical process that can require the consent, wherein the consent resource data: is generated in the second format, and includes the encrypted reference to the patient or a different encrypted reference to the patient. The method can further include causing a patient consent form to be generated based on the consent resource data, wherein the patient consent form includes natural language content data that: describes the medical process, and is at least partially based on other data stored separately from the medical records database. The method can further include causing the natural language content data to be converted to audio data using a text-to-speech process, wherein the natural language content data is converted by the medical consent application or a separate application. The method can further include causing a graphical user interface (GUI) of a client computing device to render: at least a portion of the patient consent form at an application interface of the medical consent application, and a selectable element that, in response to being selected by a patient or a patient representative, causes playback of the audio data via an audio interface of the client computing device or a separate device.

In some implementations, the method can further include causing the consent resource data to be updated to include a reference or link to the audio data subsequent to the natural language content data being converted to the audio data. In some implementations, the method can further include determining, subsequent to the GUI of the client computing device rendering the portion of the patient consent form, that playback of the audio data was performed at the client computing device or the separate device, and causing the consent resource data to be updated to indicate a duration of the audio data that was played back.

In some implementations, the method can further include generating updated consent resource data in response to the patient or the patient's providing an indication of consent to the patient consent form; and causing the updated consent resource data to be communicated, in the first format, to the medical records database for storage, wherein the updated consent resource data include a reference or link to an image that embodies the indication of consent to the patient consent form. In some implementations, the method can further include causing an updated consent form to be generated and communicated to an email address associated with the patient, wherein the updated consent form is generated as a file or image that is attached to an electronic communication that embodies another reference or other link to the image that embodies the indication of the consent to the patient consent form. In some implementations, the electronic communication further embodies a particular reference or a particular link to the audio data for playback by a recipient of the electronic communication. In some implementations, the medical process includes treatment, discharge, and/or other procedure.

In some implementations, a system is set forth as including a key value storage that stores, in a non-transitory computer-readable medium (CRM), correspondence between user identifiers and key values that can be utilized to establish secure connections between client computing devices and various medical records databases. The system can further include a consent form content storage (e.g., medical consent application data 124) that stores, in the CRM or other CRM, natural language content for consent forms, wherein the natural language content is stored in association with identifiers for respective medical procedures identified in the various medical records databases. The system can further include a medical consent application (e.g., medical consent application 114) that receives a particular user identifier of the user identifiers and causes initiation of a secure connection with a particular medical records database of the various medical records databases, wherein the particular medical records database is selected for the secure connection based on the particular user identifier. The system can further include a server application (e.g., medical consent application 114, applications 158, applications 150, applications 106, etc.) that provides executed consent forms to be communicated to the various medical records databases and electronic addresses for medical service providers, wherein the server application provides an executed consent form to an electronic address of a medical service provider in response to an executing patient indicating, via the executed consent form, that a medical provider does not have an account with the particular medical records database or is not associated with a network of the particular medical records database. In some implementations, the server application provides the executed consent form to the electronic address of the medical service provider in a consolidated clinical document architecture (CCDA) format.

In some other implementations herein, a method implemented by one or more processors is set forth as including operations such as providing a computer application accessible via a computer, wherein the computer application is in communication with a medical records system of the health care provider system and accesses electronic medical records of the patient via the medical records system. The method can further include receiving, from the patient using the computer application, an input of an identifier for the patient in furtherance of accessing patient data via the computer application. The method can further include receiving, from the patient using the computer application, a selection of a scheduled medical procedure from a menu provided by the computer application. The method can further include causing, in response to receiving the selection of the scheduled medical procedure from the menu, a procedure video with information regarding the scheduled medical procedure to be visibly rendered at an interface of the computer application. The method can further include causing, based on the selection of the scheduled medical procedure from the menu, text of a page of frequently asked questions regarding the scheduled medical procedure to be audibly rendered by the computer application. The method can further include generating, in response to the computer application visibly rendering the information regarding the scheduled medical information and audibly rendering the text of the page of frequently asked questions, informed consent information by the computer application, wherein the informed content information indicates receipt, by the patient, of the information and the text regarding the scheduled medical procedure. The method can further include causing, based on the informed consent information, the computer application to render an informed consent form at an interface of the computer application for receiving an electronic signature from the patient, wherein the informed consent form is selected from multiple digital consent forms that are stored in association with multiple different medical procedures. The method can further include receiving, by the computer application and from the patient, a drawn electronic signature of the patient, via a finger or stylus input, at an interface of the computer application. The method can further include, in response to the patient providing the drawn electronic signature via the informed consent form of the interface of the computer application: converting the drawn electronic signature into a portable document format, and sending the drawn electronic signature, embodied in the portable document format, to a health care provider and an email address of the patient. The method can further include, after the scheduled procedure: causing additional information regarding a post-procedure plan to be rendered at the interface of the computer application, wherein the post-procedure plan includes post-procedure appointment information. The method can further include determining receipt of the post-procedure plan by the patient. The method can further include, in response to the patient providing an additional electronic signature via a separate informed consent form of the interface of the computer application: sending the additional electronic signature to the health care provider, and sending the post-procedure appointment information to the patient by e-mail with an automated calendar reminder, wherein said calendar reminder is accessed by a computerized calendar of the patient to provide the patient with appointment reminders.

In some implementations, the computer application audibly renders the text based on the electronic medical records of the patient indicating that the patient is a hearing impaired patient or a patient with limited literacy. In some implementations, the method can include causing, prior to receiving the input of the identifier for the patient, the computer application to audibly instruct the patient to input a name for the patient into a field of a form of the computer application. In some implementations, the method can include causing, prior to receiving the selection of the medical procedure from the menu provided by the computer application, the computer application to audibly render names for different medical procedures from the menu.

In some implementations, the different medical procedures of the menu are selected by the computer application based on the input of the identifier for the patient. In some implementations, the computer application provides the procedure video such that the patient can stop and rewind to review procedure information provided in the procedure video. In some implementations, the identifier for the patient includes an email address for the patient, and the method further comprises: sending, by the computer application, post-procedure medication information to the patient by email to an email address of the patient. In some implementations, the additional information regarding the post-procedure plan includes procedure specific results for the patient. In some implementations, the page of frequently asked questions includes an artificial intelligence chat that can answer questions from the patient that is viewing the page of frequently asked questions. In some implementations, the computer application is a web-based application that is accessible to the patient via a web browser of the computer, and the patient signs the interface of the computer application with their finger.

The above description is provided as an overview of some implementations of the present disclosure. Further description of those implementations, and other implementations, are described in more detail below.

Other implementations may include a non-transitory computer readable storage medium storing instructions executable by one or more processors (e.g., central processing unit(s) (CPU(s)), graphics processing unit(s) (GPU(s)), and/or tensor processing unit(s) (TPU(s)) to perform a method such as one or more of the methods described above and/or elsewhere herein. Yet other implementations may include a system of one or more computers that include one or more processors operable to execute stored instructions to perform a method such as one or more of the methods described above and/or elsewhere herein.

### BRIEF DESCRIPTION OF THE DRAWINGS

FIG. 1A and FIG. 1B illustrate views of a system for providing a medical consent application that can securely access various EMR databases, having different formats, and provide consent forms that can be converted for subsequent storage at the EMR databases.

FIG. 2A, FIG. 2B, FIG. 2C, FIG. 2D, and FIG. 2E illustrate views of a patient interacting with a medical consent application that can securely interact with an EMR database while maintaining encryption of medical data.

FIG. 3A and FIG. 3B illustrate methods for providing a medical consent application that can securely retrieve EMR data, which can be stored in a variety of different formats, and render a consent form for a patient with useful information (e.g., explanatory text, audio, video, etc.).

FIG. 4 is a block diagram of an example computer system

### DETAILED DESCRIPTION

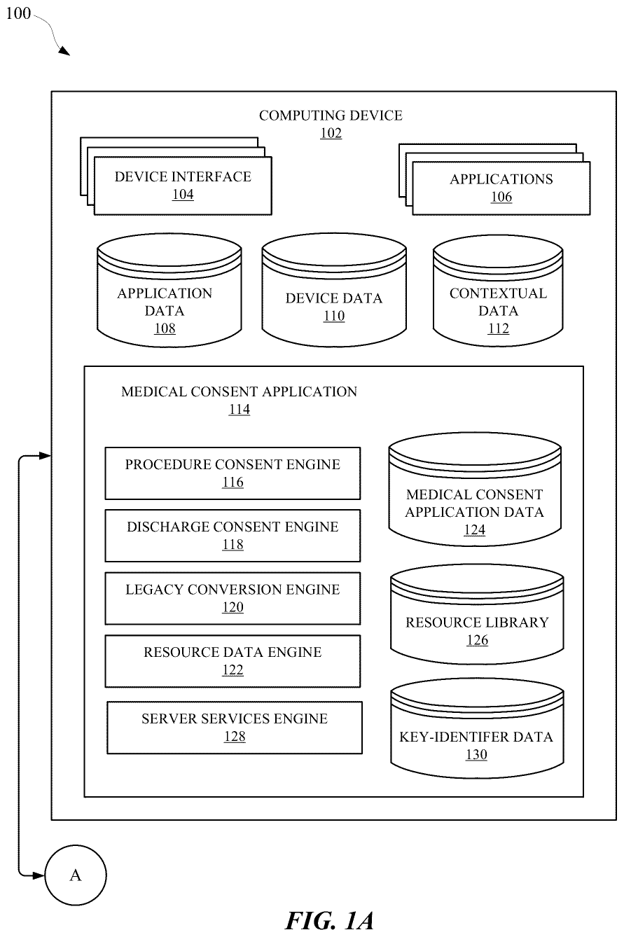

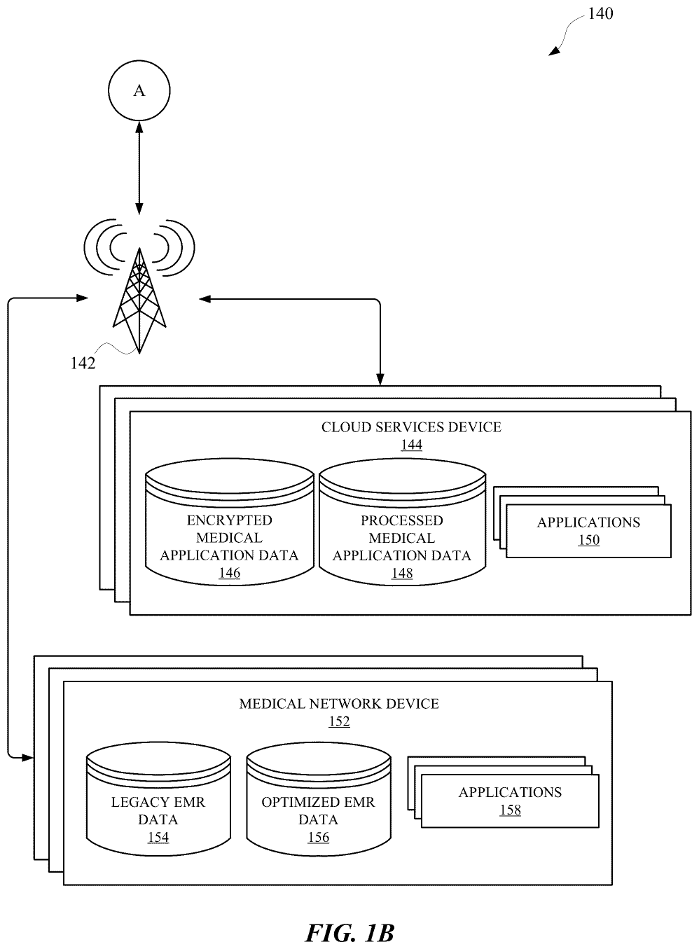

FIG. 1A and FIG. 1B illustrate a view 100 and a view 140 of a system for providing a medical consent application 114 that can securely access various EMR databases, having different formats for different provider networks, and provide consent forms that can be converted for subsequent storage at the EMR databases. The system can include a computing device 102 that can include a medical consent application 114 and can be operated by a patient, a provider of medical services, and/or any other person or entity associated with a medical provider or patient. The computing device 102 can be in communication, over one or more networks 142, with one or more cloud services devices 144 and/or medical network devices 152, as illustrated in FIG. 1A and FIG. 1B, and continuation element “A”. In some implementations, when the computing device 102 is a client computing device, the computing device 102 can include other applications 106 that can facilitate certain operations of the medical consent application 106. Additionally, the computing device 102 can include one or more different interfaces 104 for facilitating certain functionality of the medical consent application 114 and the applications 106.

In some implementations, when a user (e.g., a medical provider, a patient, etc.) accesses the medical consent application 114, the user can provide login credentials to the medical consent application. The medical consent application 114, which can be a client application and/or a server application (e.g., instances of multiple applications communicating across a network, between client(s) and server(s)), can process the login credentials to determine a correlation between the login credentials (e.g., a user identifier) and a provider network, a medical records database, and/or a medical records application. For example, the login credentials can be utilized by a server services engine 128 to determine a particular medical records database that the user is associated with and, based on this determination, select a key value and/or a specific identifier from key-identifier data 130 for establishing a secure connection with the provider network and/or provider database. In other words, the user authenticates with the medical consent application 114 and, in response, the server services engine 128 assists the medical consent application 114 with establishing a secure connection with an EMR database associated with the user. For example, the medical consent application 114 can determine that the login credentials are associated with a medical network that uses the FIHR protocol and, select a particular API key value for establishing a secure connection with the EMR database according to the FIHR protocol.

The medical consent application 114 can have access to various different medical records databases, and medical networks using various different key values, thereby allowing the medical consent application 114 to be universal across medical networks, and interface with multiple different medical records databases that embody different formats of records and/or multiple different security protocols. However, the medical consent application 114 can nonetheless provider customized and/or customizable medical consent forms. The consent forms can be compiled from data stored specifically for the medical consent application 114 and information retrieved from any of the various medical records databases that are associated with the provider that is seeking to generate the consent form. For example, portions of text of a medical consent form to be generated can be retrieved from medical consent application data 124 that can be stored at the computing device 102 and/or another computing device. Other portions of text for the medical consent form can be generated using information retrieved during token sessions with any of the accessible EMIR databases—the token sessions being established using the appropriate key value and identifier for the provider or user. In this way, a surgeon in a Texas medical network can create a consent form for heart surgery using protected EMIR data and text stored in the medical consent application data 124, while a different surgeon of a different state medical network can create a consent form for heart surgery using other protected EMR data and the text stored in the medical consent application data 124.

For example, a provider can access the computing device 102 to initialize a consent process via the medical consent application 114 in furtherance of receiving consent from a patient, or a patient's representative, for a medical process (e.g., operation, discharge, post-procedure treatment, follow-up appointments, etc.). When the provider is attempting to initialize a consent procedure for a procedure, the provider can interact with a GUI of the medical consent application 114 to initialize creation of consent resource data. The consent resource data can be generated by a procedure consent engine 116 that can be in communication with the medical network device 152 associated with the provider. When the provider creates an instance of procedure resource data using an application 158 of the medical network device 152, the procedure resource data can be stored with legacy EMR data 154. For example, the procedure can be kidney surgery, and the provider can schedule the surgery using an application 158, which can result in the creation of the procedure resource data that identifies the operation and includes an identifier for the patient.

The procedure consent engine 116 can detect changes to data associated with various patients, such as when new resource data is created with reference to a patient. The procedure consent engine 116 can determine that the procedure resource data was created with the identifier for the patient and, in response, generate an instance of consent resource data with the identifier, or a different identifier, for the patient. The consent resource data can also identify the procedure or other medical process to be consented to. Based on this procedure and/or the patient identifier, the consent resource data can include fields that are populated with values selected from medical consent application data 124, resource library 126, processed medical application data 148, legacy EMR data 154, optimized EMR data 156, and/or any other data that can be accessed by an application.

For example, in response to determining that procedure resource data has been generated for performing a particular procedure for a patient, the medical consent application 114 can determine the particular procedure and access a predefined template for consent resource data. This predefined template can be stored at the medical consent application data 124 as, for example, a filed named “Kindey_Surgery_Consent_Resource,” and can include predetermined data that can populate an interface of the medical consent application 114 when a patient is consenting to the procedure. For example, the consent resource data can include natural language content that characterizes terms of the consent and frequently asked questions (“FAQ”) about the procedure, and this natural language content can be based on text stored in the medical consent application data 124. Alternatively, or additionally, the consent resource data can include links and/or references to media, such as images, videos, and/or other media files stored in the resource library 126, which can be stored at the computing device 102 and/or at any other devices associated with the medical consent application 114. For example, a video that explains the medical procedure can be stored at the resource library 126 and/or at a third party service (e.g., hosted by a video sharing entity), thereby allowing the video to be presented to a patient when the patient is consenting to the operation via the medical consent application 114.

In some implementations, the consent resource data can be generated with information from, or references to, legacy EMR data 154 and/or optimized EMR data 156 stored at a medical network device 152, such as an EMR server or other storage device. For example, when the legacy EMR data 154 (e.g., a paper form or other non-transitory, tangible document) serves as the basis for the procedure resource data, the consent resource data can be generated to include a reference to a scanned form (e.g., a PDF, image, or other file) that indicates the provider has ordered the procedure be performed for the patient. In some implementations, fields of the consent resource data can be populated with values that are generated using an optical character recognition process performed on the scanned form. Alternatively, or additionally, the consent resource data can include data securely retrieved (with prior permission from the patient and provider) from the optimized EMR data 156, such as a patient's medical history, current medications, prior procedures, and/or any other medical information. In some implementations, the data is securely retrieved after an encrypted key exchange between the medical consent application 114 and the medical network device 152, and token generation for establishing a secure session for sharing information between the medical consent application 114 and the medical network device 152.

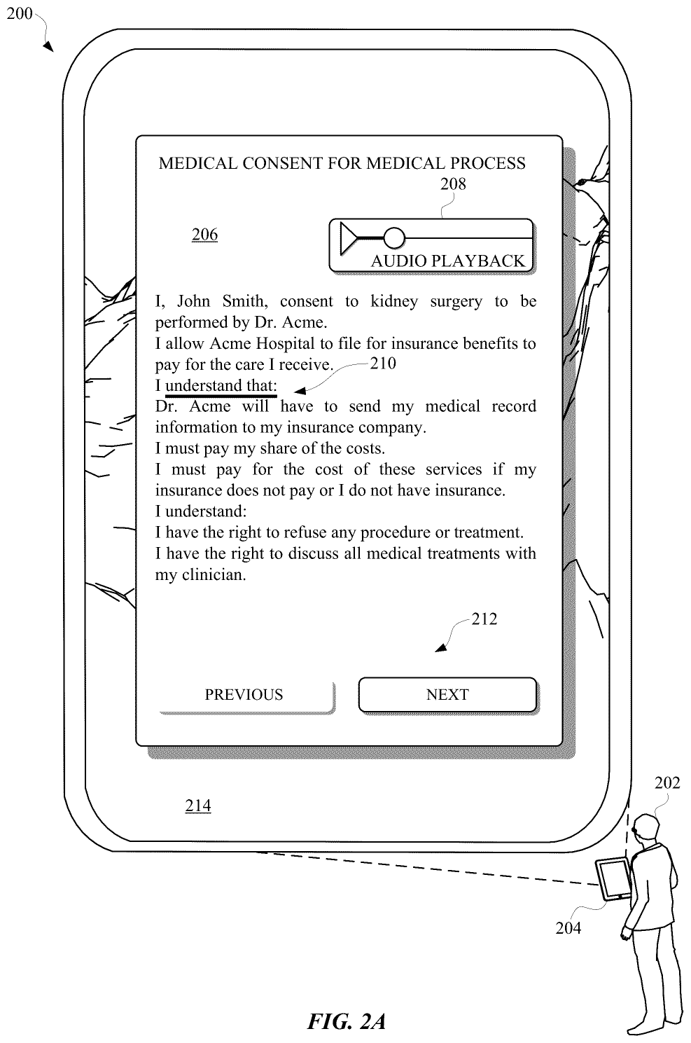

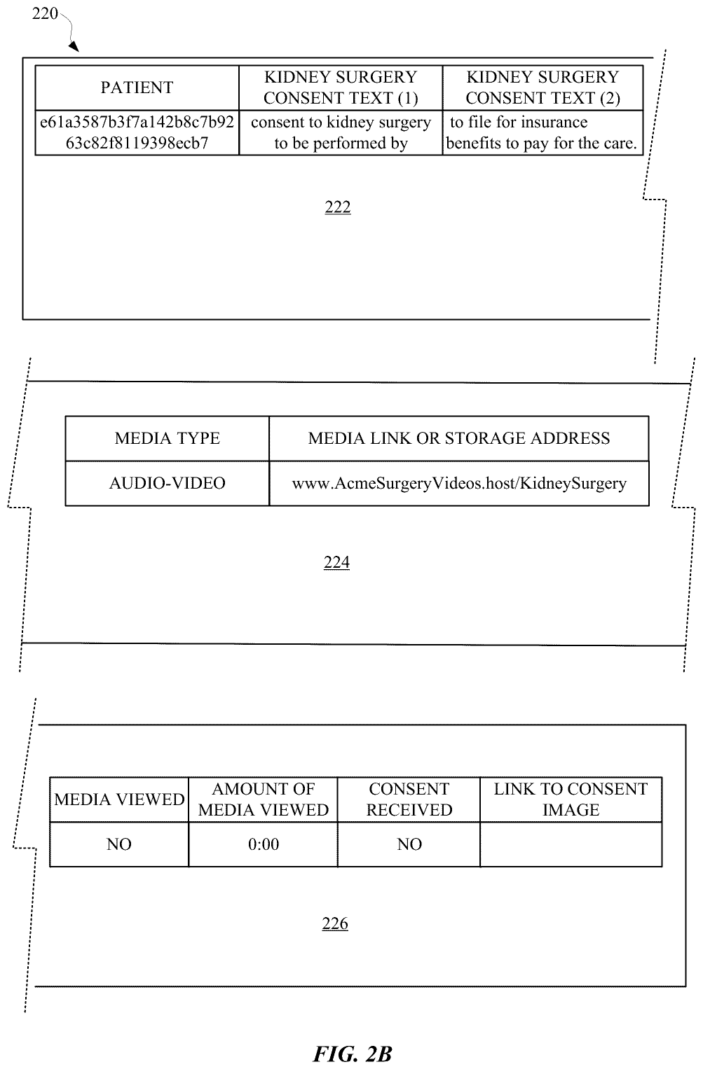

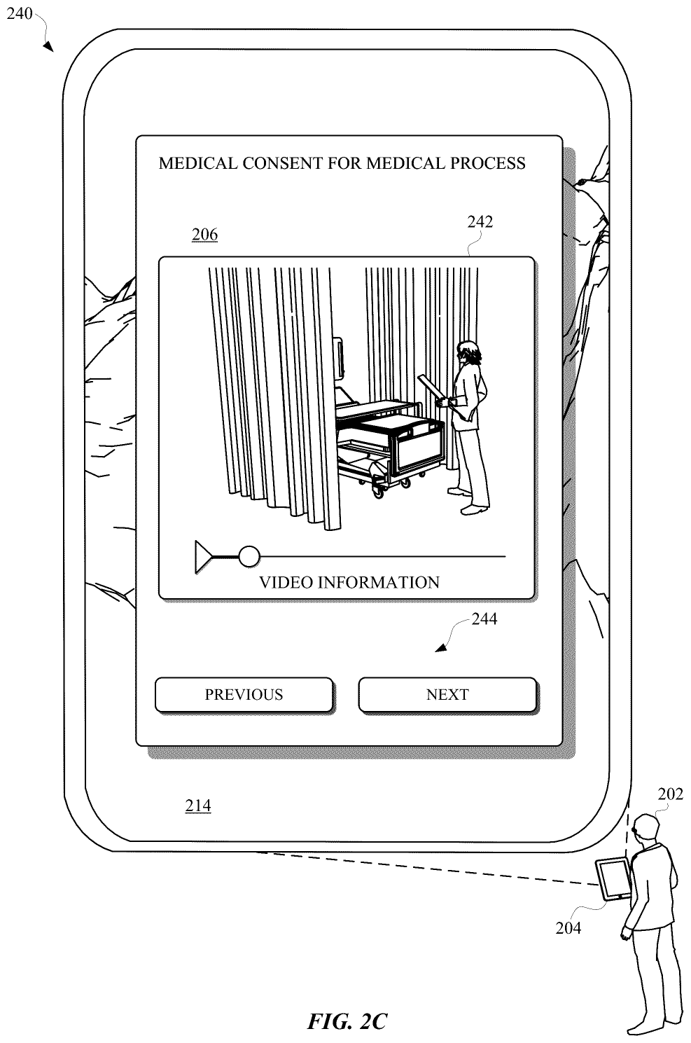

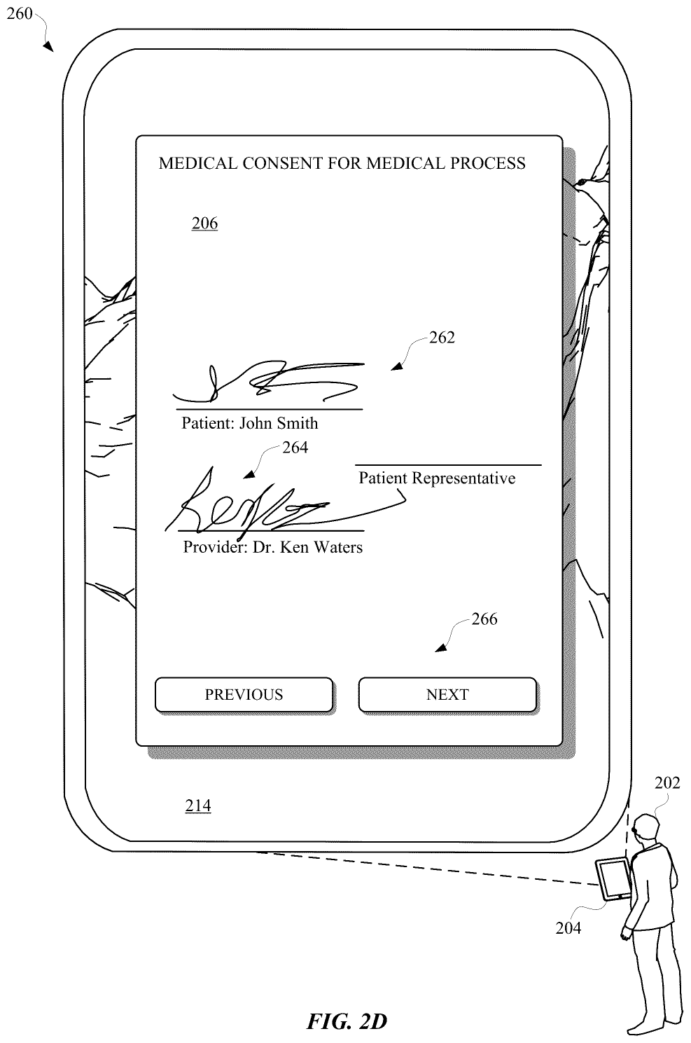

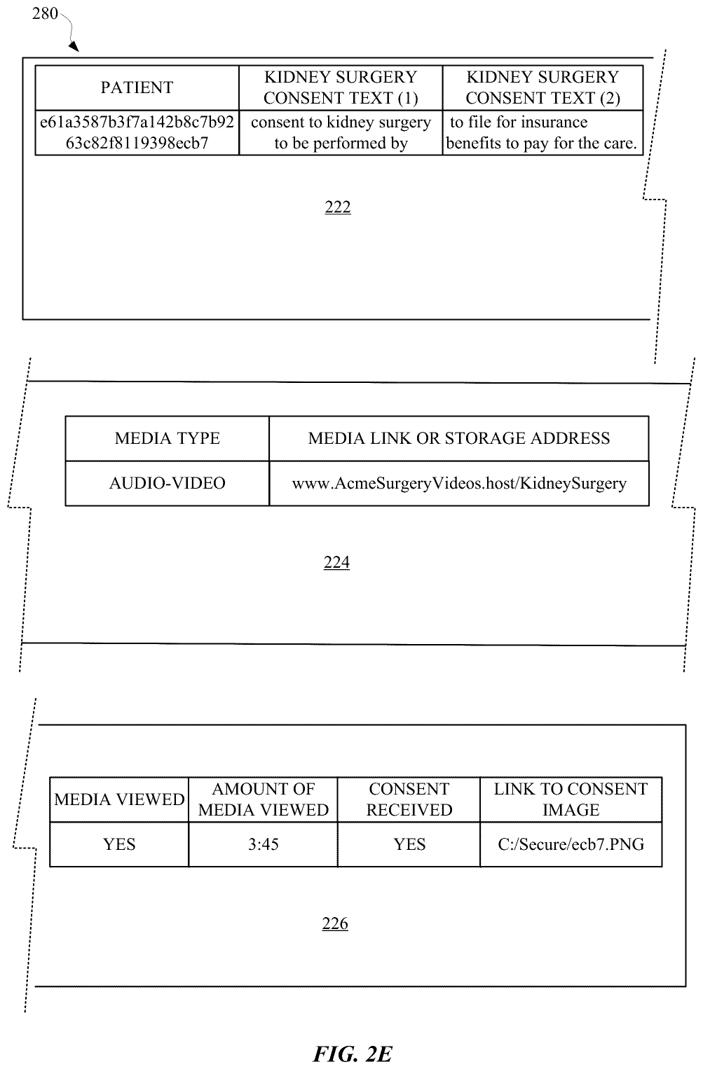

In some implementations, the EMR data retrieved for generating the consent resource data can indicate that a patient is visually impaired or has issues with hearing. Based on this data, or regardless of this data, the medical consent application 114 can solicit a cloud services application 150 to convert natural language content of the consent resource data to audio, for playback to the patient or patient's representative during a consent process. For example, the text that indicates the terms of the consent and/or description of a medical process can be communicated securely to a cloud services device 144. The received data can be stored, by the cloud services device 144, as encrypted medical application data 146 and thereafter be processed by one or more applications 150 for converting textual data to audio data. Processed medical application data 148 (i.e., the text to speech audio) generated by the one or more applications 150 can then be securely communicated back to the medical consent application 114 via the network 142 (e.g., a wireless network, WAN, LAN, etc.). In some implementations, the processed medical application data 148 can then be stored at the medical consent application 114 and/or at a medical network device 152, and a reference or link to the processed medical application data 148 can be incorporated into a field of the consent resource data. For example, an address of an audio file that includes text-to-speech audio based on text of a consent form can be included in the consent resource data. As a result, when the consent for is rendered at a GUI by the medical consent application 114, a media player can be rendered as an element of the consent form, as illustrated in FIG. 2A, thereby allowing a patient to listen to the content of the consent form.

In some implementations, the medical consent application 114 can include a discharge consent engine 118 that can generate consent resource data in response to determining that a patient is to be discharged from being hosted by a medical provider (e.g., leaving an appointment, leaving a medical facility, leaving the care of a medical provider, etc.). For example, a medical provider at a hospital can interact with the medical consent application 114 or another application 106 or application 158 to generate an instance of data indicating that a patient is to be discharged according to a particular type of discharge. In some implementations, the consent resource data that is generated by the discharge consent engine 118 can be generated based on a discharge consent template stored in the medical consent application data 124. For example, the medical consent application data 124 can include templates for being discharged after certain medical procedures, and therapies, and/or after being at a care facility and/or under the care of a medical provider (e.g., an in-home nurse, in-home physical therapist, doctor, etc.). When a template is identified for a particular type of discharge selected by the medical provider, consent resource data can be generated, based on the template, with fields and/or values corresponding to the type of discharge.

For example, when the type of discharge corresponds to a post-kidney surgery discharge, the consent resource data can include a reference or link to content stored in the resource library 126, such as videos, articles, and images, that explain post-operative care. In some implementations, when the patient or patient's representative consents to be discharged, the discharge consent engine 118 can also generate appointment resource data, which can specify an appointment and patient contact info for sending an appointment reminder. For example, content of the consent resource data can indicate that the patient should see a dietician and, in response to consenting to discharge, an instance of appointment resource data can be generated with a link or reference to a dietician, a link or reference to an application page for scheduling an appointment with the dietician, and/or contact info for the patient for communicating the dietician appointment information. Thereafter, when the patient has completed the discharge process with the medical consent application 114, the medical consent application 114 can cause the appointment resource data to be processed for rendering an appointment scheduling interface. The consent resource data and/or the appointment resource data can then be updated and communicated back to the medical network device 152 for storage at the optimized EMR data 156. In some implementations, an application 158 of the medical network device 152 or the medical consent application 114 can periodically process resource data that identifies or otherwise references the patient to determine reminders or other correspondence that should be automatically communicated to the patient (e.g., medication reminders, therapy reminders, appointment reminders, etc.).

In some implementations, the optimized EMR data 156 can be stored in a first format that is different from a second format of the medical consent application data 124 of the medical consent application 114. In some implementations, the legacy EMR data 154 can be stored in an image format that has not been parsed into any other type of data structure (e.g., JSON, XML, etc.). In such instances, a legacy conversion engine 120 of the medical consent application 114 can process data (e.g., provider patient data) from the medical network device 152 and convert this data into the second format. In this way, data from the medical consent application data 124 and the resource library 126 can be combined with data from any EMIR database to efficiently generate custom forms per network provider, type of discharge, and type of consent to a medical process. For example, when a token session has been established with the medical network device 152, the legacy conversion engine 120 can convert medical information associated with a particular patient from a first format (e.g., including but not limited to JSON) to a second format (e.g., including but not limited to SQL). Initially, the first format data can be encrypted and communicated over the network 142, and then dencrypted by the medical consent application 114 and converted into the second format. In some implementations, “encrypted” can refer to a modification to data that can cause the data to be different from an original instance of the data, for the intention of obfuscating an original meaning, source, and/or value of the original instance of the data from someone who does not have permission to access the original instance of the data. Such retrieval and conversion can be performed, for example, in response to a patient or a medical provider accessing the medical consent application 114 to initialize or complete a particular type of consent process. Depending on the type of consent process, the EMR data converted to the second format can be incorporated into consent resource data that includes other content according to type of consent selected by the medical provider and/or patient.

When the patient or patient's representative has completed the consent process, the consent resource data can be converted from the second format to the first format, encrypted, and then communicated back over the network 142 to the medical network device 152. The updated consent resource data that is communicated back to the medical network device 152 can include an indication of the patient's consent, such as via a value, and/or link or other reference to an image file embodying the patient's signature, the patient's representative's signature, and/or a provider's signature. In some implementations, the updated consent resource data can also include links or references to materials viewed by the patient, the patient's representative, and/or medical provider during the consent process and/or during other process associated with providing the consent. For example, the consent resource data can include a link or reference to an explanatory video along with an indication of an amount of the video that the patient or patient's representative watched (e.g., 100%, 15 minutes and 30 seconds of 16 minutes, etc.). In some implementations, a file representing the consent form that embodies some or all of the data in the consent resource data can be generated by the medical consent application 114 and communicated to the patient, patient's representative, medical provider, and/or any other address for any other person or entity associated with the patient or medical process. In some implementations, this operation can be performed by the medical consent application 114 and/or by an application 158 of the medical network device 152 in response to the consent process being completed at the medical consent application 114 or the medical network device 152 receiving the consent resource data that indicates the consent process was completed. This communication of the executed/signed consent form and/or the consent resource data can be performed in response to completion of the medical procedure consent, discharge consent, and/or any other type of consent that can be associated with a patient or user of an application.

In some implementations, the medical consent application 114 can include a resource data engine 122 that can generate each instance of resource data and/or receive inputs from users (e.g., providers, patients, etc.) for defining other types of resource data or content of resource data. For example, a provider can access the medical consent application 114 via a web browser (e.g., as a web app) or as a standalone application, and can provide inputs that can be used to generate content to be stored in the medical consent application data 124 and/or the resource library 126. In some implementations, a patient can provide questions to the medical consent application 114 about a particular consent process or procedure and, when a threshold number of patients have provided a particular question, the medical consent application 114 can store the question as part of a “FAQ” that can be stored in the resource library 126. Thereafter, one or more resource data templates associated with that particular medical consent process or procedure can include a reference or link to a document that includes the FAQs from various patients. Alternatively, or additionally, a provider can generate content such as video, text, or audio using the computing device 102, and the content can be processed by the resource data engine 122 and stored in association with any particular consent resource data template that the provider may desire.

For example, the provider may want to record audio, video, or text to be included in content that a patient views when consenting to a kidney surgery procedure. The provider can utilize the medical consent application 114 to record an explanatory video and the resource data engine 122 can process the video and convert the video into a format (e.g., convert to a compressed video, extract audio only, extract text and convert to a text file, etc.) that can be stored as a file that can be linked or referenced from a kidney surgery consent resource data template. Thereafter, when another patient is to undergo kidney surgery by that provider, the kidney surgery consent resource data template can be selected and populated with the other patient data, along with a reference or link to the video that the provider created with the medical consent application 114.

In some implementations, modification to consent resource data can affect other resource data, such as procedure resource data. For example, should a patient refuse to consent to a medical procedure, the consent resource data can be modified to indicate that the patient refused to consent. In response to the consent resource data being updated to indicate the refusal, the medical consent application 114 can cause procedure resource data to be modified at the medical network device 152. For example, in response to the refusal, the medical consent application 114 can initialize a secure session with the medical network device 152 to modify or delete the procedure resource data generated by an application 158 of the medical network device 152 (with prior permission from the provider and/or the patient). In some implementations, the medical consent application 114 can determine a format of the data stored at the medical network device 152 based on establishing the secure session and, based on this format, generate any requests or communications for the medical network device 152 according to the identified format.

FIG. 2A, FIG. 2B, FIG. 2C, FIG. 2D, and FIG. 2E illustrate a view 200, a view 220, a view 240, a view 260, and a view 280 of a patient 202 interacting with a medical consent application that can securely interact with an EMR database while maintaining encryption of medical data. For example, FIG. 2A illustrates a view 200 of an interface of the medical consent application being rendered at a display interface 214 of a computing device 204. The medical consent application can generate a consent form in response to determining the patient 202 is to undertake a medical process, such as undergoing to a medical procedure, being discharged from a medical facility, and/or any other suitable medical process. In some implementations, the medical consent application can store data associated with certain medical processes and, in response to determining that the patient 202 is to undergo the medical process, the medical consent application can access the data.

In some implementations, the data accessed by the medical consent application can be stored in various forms such as, but not limited to a SQL database, JSON, XML, and/or any other form of data organization. The data can include fields with values that indicate descriptions of certain medical processes, links or references to medical processes, links or references to images of a signature of a patient or patient's representative, an amount of a form or media that has been viewed by a patient or representative, and/or any other data that can be associated with a medical process. In some implementations, an instance of data can be parsed in response to a request by the medical consent application in furtherance of generating an interface, form, or other media for the medical consent application. For example, and as provided in FIG. 2A, the medical process that the patient 202 is to undergo can be a kidney surgery, and a provider (e.g., a kidney doctor) can solicit the medical consent application to generate a form for the patient to consent to the kidney surgery.

For example, in response to the provider providing an input to the medical consent application to generate a consent form 206, the medical consent application can establish a secured connection with an EMR database. In some implementations, the medical consent application can provide a key value to a device hosting the EMR database in furtherance of receiving a token that allows the medical consent application to have a secured session with the EMR database. When the secured session is initiated with the EMR database, the medical consent application can retrieve an identifier for the patient, which can be encrypted for usage at the medical consent application. In some implementations, the retrieved data can be converted from a first format of the EMR database to a second format (different from the first format) of resource data available via the medical consent application. For example, the EMR database can store procedure resource data that can include a field identifying the procedure to be performed on the patient 202, the doctor to perform the procedure, a date and/or estimated duration scheduled for the procedure, and/or any other information that can be specified by a provider.

The procedure resource data can be converted into the second format and utilized to retrieve other resource data available to the medical consent application. For example, the medical consent application can store, or otherwise access, patient resource data and/or procedure resource data to generate an instance of consent resource data, which can provide a basis for generating the consent form 206. The consent resource data can be illustrated at view 220 of FIG. 2B, which can show a first portion 222, a second portion 224, and a third portion 226 of the consent resource data. For example, a patient identifier can be an encrypted reference to the patient 202 and/or patient resource data stored at the EMR database. Additionally, the consent resource data can include natural language content retrieved from a database of the medical consent application in furtherance of incorporating them into the consent form 206. For example, “consent to kidney surgery to be performed by” can be natural language content that is to be included in the consent form 206, and that is stored by the medical consent application and/or another device accessible to the medical consent application (but different from the EMIR database). In some implementations, the consent resource data can be formatted based on a particular medical consent template to include certain fields, and therefore certain content for the form, such as “audio-video,” a kidney surgery video, and text requesting that the patient's insurance be notified of the procedure. As the patient 202 continues to interact with the consent form 206, the values of the fields of the consent resource data can be updated dynamically.

For example, when the patient selects a selectable element to initialize audio playback 208 of the consent form 206, a value of the consent resource data table can be updated to include an indication of how much audio of the consent form 206 has been played. In some implementations, in response to the consent form 206 being generated and/or the consent resource data being generated, the consent resource data and/or natural language content of the consent form 206 can be converted to audio. For example, the natural language content of the consent form 206 can be securely communicated to a third party text-to-speech service (with prior permission from the patient 202 and the provider), which can send an audio file back to the medical consent application. As the audio is played back for the patient 202, a dynamic element 210 can be rendered at the consent form 206 to indicate the portion of the consent form 206 that is being read aloud via audio playback 208 of the audio file. In some implementations, the audio playback can cause the consent form 206 to show different pages as audio for a single page has been read about to the patient 202. Otherwise, the patient 202 or a representative of the patient can select a selectable element 212 to cause the medical consent application to render a proceeding page of the generated consent form 206.

Another page of the consent form 206 generated by the medical consent application can include a video player 242 for playing an explanatory video for a patient 202 or representative, as illustrated in view 240 of FIG. 2C. As the patient 202 reviews the video played by the video player 242, the consent resource data can be updated to reflect how much of the video the patient has viewed during the consent process, as illustrated in FIG. 2E at the third portion 226 of the consent resource data (e.g., “Amount of Media Viewed: 3:45”). In this way, the consent resource data can be communicated with such indications, thereby allowing the provider and patient to reference the data that was reviewed by the patient 202, or the representative, in preparation for a medical process. When the patient 202 has completed viewing the video at the video player 242, the patient 202 can select a selectable element 244 for viewing another page of the consent form 206, such as a page for providing a signature, as illustrated in FIG. 2D.

In some implementations, a number of fields for signatures can correspond to a number of fields 262 of the consent resource data that are available for storing references to signatures. The patient 202 and provider can provide their signatures 264 by providing a direct touch input to the display interface 214 of the computing device 204, by typing in their signatures, and/or by otherwise indicating their consent via the medical consent application. In some implementations, in response to providing their signatures, values for fields of the consent resource data can be generated and stored in random access memory of the computing device 204, before being communicated back to the EMR database. For example, and as illustrated in view 280 of FIG. 2E, a final form of the consent resource data after a patient 202 completed a consent process for a medical process can include a reference to any audio and/or video file that was viewed during the consent process (as shown in the second portion 224), an indication that the media was viewed (as in the third portion 226), an encrypted reference to the patient or the patient resource data (as in the first portion 222), text of the consent form (as in the first portion 222), and references and/or links to signatures of the patient, provider, and/or patient representative (as shown in the third portion 226 as “C:Secure/ecb7.png”).

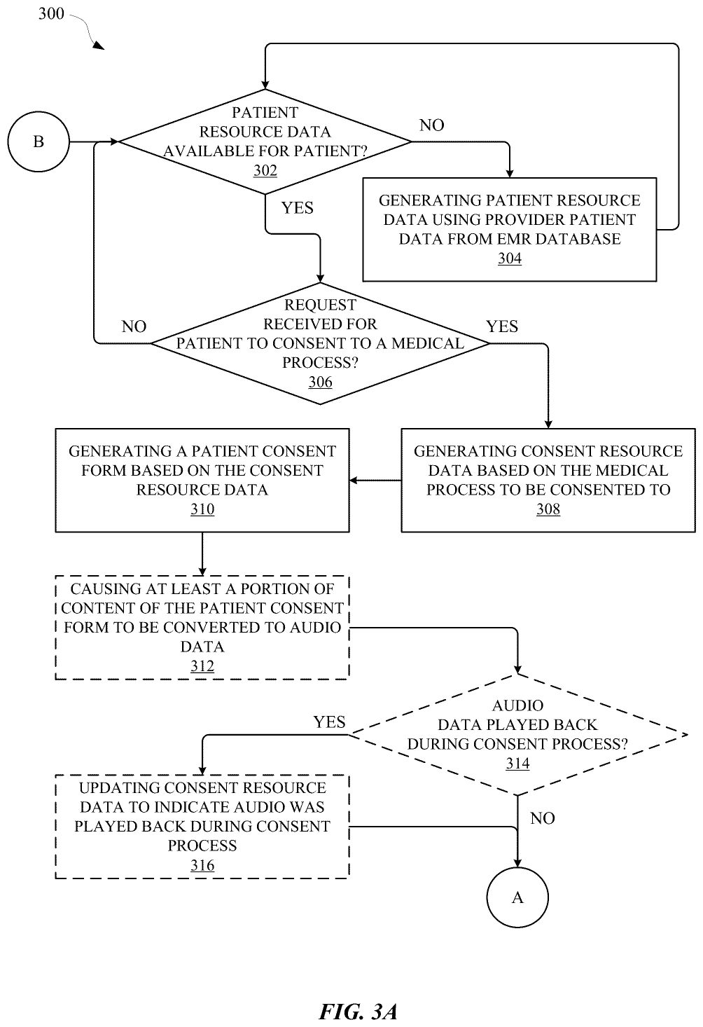

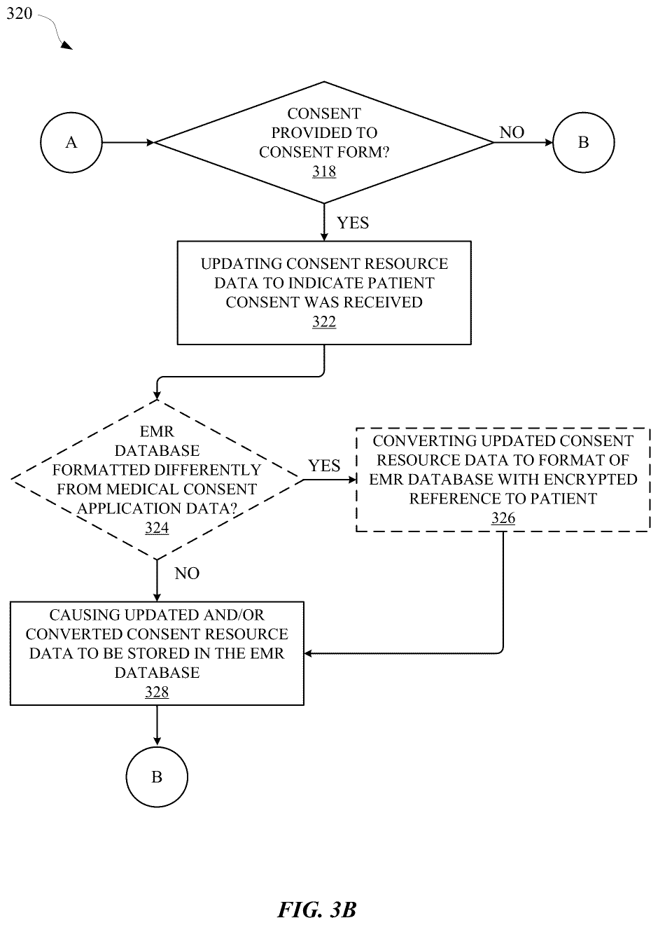

FIG. 3A and FIG. 3B illustrate a method 300 and a method 320, respectively, for providing a medical consent application that can securely retrieve EMR data, which can be stored in a variety of different formats, and render a consent form for a patient with useful information (e.g., explanatory text, audio, video, etc.). An executed consent form can then be converted into a secure format for communicating back to the EMR database, without risking privacy of any associated patients. The method 300 can be performed by one or more computing devices, applications, and/or any other apparatus or module that can be associated with a medical application and/or network-enabled device. The method 300 can include an operation 302 of determining whether patient resource data is available for a patient. The patient can be accessing a medical consent application for consenting to discharge, a medical treatment, and/or another medical process. The patient resource data can be data that provides an encrypted reference to a patient and/or to an address of an EMR record and/or an EMR database. In some implementations, the patient resource data can be formatted as JSON, XML, SQL, and/or another data format for organizing data.

When patient resource data is available for a patient that is to be consenting to a medical process, the method 300 can proceed from the operation 302 to an operation 306. Otherwise, when no patient resource data is available for the patient, the method 300 can proceed from the operation 302 to an operation 304. The operation 304 can include generating patient resource data using provider patient data from an EMR database. In some implementations, the provider patient data can be securely managed by a hospital, medical provider, or other associated entity. Accessing the provider patient data can involve a medical consent application providing a secure request to an EMR application that manages the EMR database, and the request can include credentials that have been previously approved by an entity that manages the EMR database. In response to receiving the request, the EMR application can respond with information about the medical process that the patient is being requested to consent to, and encrypted patient information (with prior permission from the patient or a representative of the patient). The medical consent application can then process the received information and encrypted information to generate the patient resource data. The method 300 can then proceed from the operation 304 and return to the operation 302.

The operation 306 can include determining whether a request has been received for the patient to consent to a medical process. For example, the patient can be requested to consent to a medical treatment, a discharge process, a follow-up appointment, post-procedure care, to receive medication reminders, and/or any other process that can be associated with medical care. In some implementations, the request can be generated by the same entity that also manages the EMR database, or a different entity that is otherwise associated with a medical provider for the patient. In some implementations, the request for consent can be generated in response to a provider (e.g., the patient's primary care doctor) interacting with the medical consent application before or after a medical process. For example, the medical provider can securely log into the medical consent application and request that a patient consent form be generated for a particular medical process that the medical provider expressly identifies via interaction with the medical consent application.

When a request is received for a patient to consent to a medical process, the method 300 can proceed from the operation 306 to an operation 308. Otherwise, the method 300 can proceed from the operation 306 to the operation 302. The operation 308 can include generating consent resource data based on the medical process to be consented to. In some implementations, the consent resource data can be formatted according to a protocol for formatting the patient resource. The consent resource data and the patient resource can each include a reference to the patient that is encrypted and/or is otherwise embodied as a secure link to the provider patient data. Alternatively, or additionally, the consent resource data can include a reference (e.g., an identifier or link) to the patient resource data for correlating the instance of patient resource data to the instance of the consent resource data.

In some implementations, the consent resource data can also identify textual content, video content, and/or graphical content to be rendered by the medical consent application during a consent process for consenting to the medical process. For example, the consent resource data can include multiple different fields, each with an instance of data. A particular field can be designated for linking to a video that can be rendered by the medical consent application during the consent process. Alternatively, or additionally, another particular field of the consent resource data can include natural language content (e.g., English language, Chinese language text, etc.) that can describe the medical process that the patient, or patient's representative, is consenting to. In some implementations, the natural language content can be input by the medical provider via an input interface of the medical consent application, thereby allowing the medical provider or another entity associated with the medical provider to manually create portions of content for consent forms. For example, the medical provider can utilize the medical consent application to create a new medical procedure (e.g., a type of orthopedic surgery) and type in descriptions of the procedure, as well as descriptions of how to prepare for, and recover from, the new medical procedure. Alternatively, or additionally, the natural language content can be generated by an artificial intelligence application (e.g., an application employing a large language model, or other language model) to describe the medical process and/or the consent process.

The method 300 can proceed from the operation 308 to an operation 310, which can include generating a patient consent form based on the consent resource data and/or the patient resource data. In some implementations, the operation 310 can include arranging data embodied in, or referenced by, the consent resource data and/or the patient resource data into a format that, when accessed by the medical consent application, will appear as a consent form. For example, an identifier for the patient can be stored such that the identifier appears at the beginning and/or the end of the consent form. Alternatively, or additionally, the natural language content describing the medical process can be formatted to appear in a body of the consent form. Alternatively, or additionally, a link to a video file can be embodied in the consent form with a reference to a preferred media player. This can allow the medical consent application to initialize the preferred media player with the video in response to the consent form being rendered at a graphical user interface (GUI) of the medical consent application. For example, the medical application can be at least partially coded in Javascript, or any other programming language, and code for rendering the consent form can include references to entries in a SQL table, or other collection of data, for providing the video and other content for the consent form. Alternatively, or additionally, code for the GUI can also reference locations in the EMR database of the medical provider, with prior permission from patient and the medical provider.

The method 300 can proceed from the operation 310 to an optional operation 312, which can include causing at least a portion of content of the patient consent form to be converted to audio data. In some implementations, the operation 312 can include communicating content of the consent form, with prior permission from the patient and the medical provider, to an application for performing a text-to-speech operation. In some implementations, the text-to-speech application can be a third party application (e.g., third party relative to a provider of the medical consent application), such as a cloud service provider that processes the received content at a remote server and communicates any resulting data (e.g., an audio file, video file, etc.) back to the medical consent application. The text-to-speech operation can involve converting content, that is based on the provider patient data, the patient resource data, and/or the consent resource data, into audio that can be played back for a patient or a patient's representative. When the medical consent application receives the resulting audio file, the medical consent application can cause an audio playback element to be rendered at a GUI of each page of the consent form. In this way, the patient or patient's representative can choose to have any portion of the consent form read back to them, which can eliminate the need for a provider to read the consent form to them and otherwise provide the consent form via various modalities for more effectively communicating medical information to a consenting patient.

The method 300 can proceed from the operation 312 to an optional operation 314, which can include determining whether audio data was played back during the consent process for the patient. In other words, the medical consent application and/or another application can determine whether the patient or patient's representative caused playback of the audio generated in the operation 312. For example, the medical consent application can determine how much of, or a percentage of, available audio of the consent form was played back during the consent process. The method 300 can proceed from the operation 314 to an optional operation 316 when the audio data was played back during the consent process. The operation 316 can include updating the consent resource data to indicate audio was played back during the consent process. In other words, the consent resource data can include a field that indicates whether the patient or the patient's representative caused playback of the audio data during the consent process, and, optionally, an amount of audio that was played back during the consent process. In some implementations, the consent resource data can also be updated to include a field that references or links to the audio data the embodies the text-to-speech audio characterizing the content of the consent form.

The method 300 can proceed from the operation 316, or from the operation 314, via continuation element “A”, to an operation 318 provided in method 320 of FIG. 3B. The operation 318 can include determining whether consent was provided at the consent form. Consent can be provided by the patient, the patient's representative, a medical provider, and/or any other entity that can be associated with a medical process, and each respective consent can be indicated at the consent form and/or the consent resource data. Each instance of consent can be provided to the medical consent application via a touch interface of a client computing device that provides access to the medical consent application and/or via another device that is in communication with the client computing device, such as a Bluetooth or Wi-Fi enabled device. Alternatively, or additionally, consent can be determined from an imaging processing technique performed by the medical consent application on a tangible printed form that can be scanned using a camera of the medical consent application. Alternatively, or additionally, consent can be determined from an image processing technique and/or audio processing techniques performed by the medical consent application on images and/or audio captured by the client computing device, with prior permission from the patient (e.g., audio can be captured when a patient is experiencing issues with hearing or seeing, and/or images can be captured when a patient is experiencing issues with hearing or seeing).

When the consent is received at the consent form, the method 320 can proceed from the operation 318 to an operation 322. Otherwise, the method 320 can return to the operation 302, or another operation, via continuation element “B.” The operation 322 can include updating the consent resource data to indicate patient consent was received. In some implementations, the consent resource data can be updated to include a field value that specifies that consent was received at the consent form. Alternatively, or additionally, the consent resource data can be updated to include a field value with a link or reference to an image of the signatures or signatures of the patient, the patient's representative, the medical provider, and/or any other associated entity. In some implementations, the consent form can be converted into a document file that can be stored at the EMR database, or another associated storage device, and the consent resource data can be updated to include a reference or link to the document file. The document file can include graphical renderings of the signatures of anyone who provided their signatures to the consent form.

The method 320 can proceed from the operation 322 to an optional operation 324, which can include determining whether the EMR database is formatted different from the medical consent application data. For example, the EMR database can store data in a first format and the medical consent application data can be stored in a second format that is different from the first format. Alternatively, or additionally, the EMIR database can be encrypted according to a first protocol and the medical consent application data can be encrypted according to a second protocol that is different from the first protocol. As a result, the medical consent application can perform data formatting on the updated consent resource data to ensure that the data is received at the EMR database or other device in an operable format for the destination.

When the EMR database is formatted differently from the medical consent application data, the method 320 can proceed from the operation 324 to an operation 326. Otherwise, the method 320 can proceed from the operation 324 to an operation 328. The operation 326 can include converting the updated consent resource data to a format of the EMR database. In some implementations, the updated consent resource data can include an encrypted reference to the patient and/or an address of provider patient data stored at the EMR database. In this way, the medical consent application can ensure secure transmission of patient data without comprising security of any parties involved in the consent process. Otherwise, when the EMR database is formatted similar to the medical consent application data, the updated consent resource data can be communicated to the EMR database and can optionally include the encrypted reference to the patient and/or an address of provider patient data. The method 320 can optionally proceed from the operation 328, via continuation element “B,” back to the operation 302 or another operation of the method 300, the method 320, and/or any other operation suitable for performing a consent process.

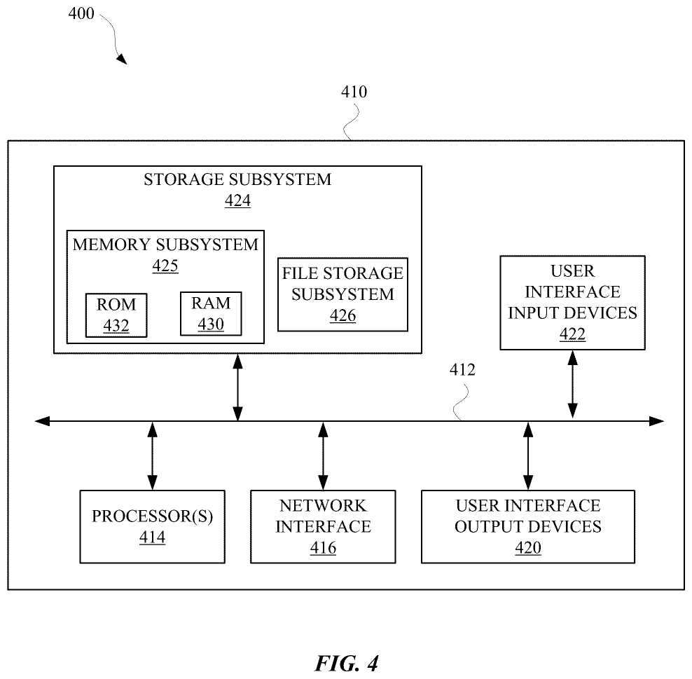

FIG. 4 is a block diagram 400 of an example computer system 410. Computer system 410 typically includes at least one processor 414 which communicates with a number of peripheral devices via bus subsystem 412. These peripheral devices may include a storage subsystem 424, including, for example, a memory 425 and a file storage subsystem 426, user interface output devices 420, user interface input devices 422, and a network interface subsystem 416. The input and output devices allow user interaction with computer system 410. Network interface subsystem 416 provides an interface to outside networks and is coupled to corresponding interface devices in other computer systems.

User interface input devices 422 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 410 or onto a communication network.

User interface output devices 420 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 410 to the user or to another machine or computer system.

Storage subsystem 424 stores programming and data constructs that provide the functionality of some or all of the modules described herein. For example, the storage subsystem 424 may include the logic to perform selected aspects of method 300 and method 320, and/or to implement one or more of system of FIG. 1A and FIG. 1B, computing device 104, a medical consent application, and/or any other application, device, apparatus, and/or module discussed herein.

These software modules are generally executed by processor 414 alone or in combination with other processors. Memory 425 used in the storage subsystem 424 can include a number of memories including a main random access memory (RAM) 430 for storage of instructions and data during program execution and a read only memory (ROM) 432 in which fixed instructions are stored. A file storage subsystem 426 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 426 in the storage subsystem 424, or in other machines accessible by the processor(s) 414.

Bus subsystem 412 provides a mechanism for letting the various components and subsystems of computer system 410 communicate with each other as intended. Although bus subsystem 412 is shown schematically as a single bus, alternative implementations of the bus subsystem may use multiple busses.

Computer system 410 can be of varying types including a workstation, server, computing cluster, blade server, server farm, or any other data processing system or computing device. Due to the ever-changing nature of computers and networks, the description of computer system 410 depicted in FIG. 4 is intended only as a specific example for purposes of illustrating some implementations. Many other configurations of computer system 410 are possible having more or fewer components than the computer system depicted in FIG. 4.

In situations in which the systems described herein collect personal information about users (or as often referred to herein, “participants”), or may make use of personal information, the users may be provided with an opportunity to control whether programs or features collect user information (e.g., information about a user's social network, social actions or activities, profession, a user's preferences, or a user's current geographic location), or to control whether and/or how to receive content from the content server that may be more relevant to the user. Also, certain data may be treated in one or more ways before it is stored or used, so that personal identifiable information is removed. For example, a user's identity may be treated so that no personal identifiable information can be determined for the user, or a user's geographic location may be generalized where geographic location information is obtained (such as to a city, ZIP code, or state level), so that a particular geographic location of a user cannot be determined. Thus, the user may have control over how information is collected about the user and/or used.

While several implementations have been described and illustrated herein, a variety of other means and/or structures for performing the function and/or obtaining the results and/or one or more of the advantages described herein may be utilized, and each of such variations and/or modifications is deemed to be within the scope of the implementations described herein. More generally, all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific implementations described herein. It is, therefore, to be understood that the foregoing implementations are presented by way of example only and that, within the scope of the appended claims and equivalents thereto, implementations may be practiced otherwise than as specifically described and claimed. Implementations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

We claim:

1. A method implemented by one or more processors, the method comprising:
receiving, at a medical consent application of a client computing device, user credentials for providing a user with access to provider-specific functionality of the medical consent application,
wherein the medical consent application provides functionality for multiple different medical providers;
determining, based on the user credentials, a key value and a provider-specific identifier that are stored in association with the user credentials by the medical consent application or another device associated with the medical consent application;
establishing, using the key value and the provider-specific identifier, a secure connection between the medical consent application and the separate computing device that provides access to a medical records database,
wherein establishing the secure connection includes providing the key value and the provider-specific identifier to the separate computing device to cause the separate computing device to share a token value with the medical consent application;
determining, by the medical consent application or another application, a format of a plurality of formats that the medical records database stores provider patient data for a patient of the user;
generating, based on the format for the medical records database, patient resource schema data using the provider patient data,
wherein the provider patient data is accessed during a token session between the medical consent application and the separate computing device, and
wherein the patient resource schema data: is generated in a separate format from the format of the provider patient data, and includes a field with a value for an encrypted reference to the patient;
determining, based on the provider patient data, that the patient is scheduled to undergo a medical procedure that can require consent from the patient, or an authorized representative of the patient, before the medical procedure is initiated by a provider or the user;
accessing, by the medical consent application and based on the medical procedure, procedure schema data that is stored in the separate format,
wherein various medical procedure schema data is accessible to the medical consent application to generate medical consent forms for multiple different medical procedures for the multiple different medical providers;
generating, by the medical consent application and based on the procedure schema data, consent resource schema data to be generated in response to determining that the patient is scheduled to undergo the medical procedure that can require the consent,
wherein the consent resource schema data: is generated in the separate format, identifies the medical procedure, and includes the encrypted reference to the patient or a different encrypted reference to the patient;
causing, by the medical consent application, a patient consent form to be rendered at a display interface of the client computing device using the consent resource schema data,
wherein the patient consent form includes natural language content embodied in fields of the procedure schema data and that describes the medical procedure;
determining that the patient, or the authorized representative of the patient, signed the patient consent form using the medical consent application or a separate application,
wherein the patient consent form is signed via direct input to the display interface of the client computing device or other client device; and
generating, by the medical consent application, updated consent resource schema data in response to determining that the patient consent form was signed,
wherein the updated consent resource schema data includes another encrypted reference to the patient and indicates the patient consent form was signed;
converting the updated consent resource schema data into formatted consent resource data,
wherein converting the updated consent resource schema data includes changing the separate format of the updated consent resource data to the format of the medical records database; and
providing, by the medical consent application, the formatted consent resource data to the separate computing device during the token session or a subsequent token session between the client computing device and the separate computing device.

2. The method of claim 1, further comprising:
determining that the user or a medical services provider has caused a particular instance of procedure resource data to be generated,
wherein determining that the patient is scheduled to undergo the medical procedure that can require consent from the patient, or the authorized representative of the patient, is performed in response to determining that the medical services provider has caused the particular instance of procedure resource data to be generated.

3. The method of claim 1, wherein the provider patient data is stored in the medical records database as one or more PDF, TIFF, JPEG, PNG, and/or BMP files, and the patient resource schema data is stored separately from the medical records database in a JSON, SQL, and/or XML format.

4. The method of claim 1, wherein generating the consent resource data includes:
causing the consent resource schema data to include a consent field with a first consent value that indicates the patient has not consented to the medical procedure,
wherein a second consent value is provided in another consent field in the updated consent resource schema data and the second consent value provides an indication that the patient, or the authorized representative of the patient, consented to the medical procedure.

5. The method of claim 4, wherein generating the consent resource schema data includes:
causing the consent resource data to include a media field with a media value that provides a link to, and/or a description of, medical procedure information to be presented to the patient, or the authorized representative of the patient, via the interface of the client computing device.

6. The method of claim 5, wherein generating the consent resource data includes:
causing the consent resource schema data to include a media acknowledgement field with a first acknowledgement value that indicates the medical procedure information has not been accessed,
wherein a second acknowledgement value is provided in another media acknowledgement field in the updated consent resource schema data, and the second acknowledgement value indicates the medical procedure information was accessed.

7. The method of claim 6, wherein the second acknowledgment value is embodied in the formatted consent resource data that is provided to the separate computing device.

8. A method implemented by one or more processors, the method comprising:
determining, by a medical consent application that is accessible via a client computing device, that a patient is to be presented with a consent form for consenting to a medical procedure,
wherein the patient is identified in patient resource data that is stored in a medical provider computing device that is separate from the client computing device;
causing, in response to determining that the patient is to be presented with the consent form, consent resource data to be generated,
wherein the consent resource data identifies the medical procedure and includes an encrypted reference to the patient resource data that identifies the patient;
determining that the patient, or an authorized representative of the patient, provided express consent for the medical procedure at the consent form,
wherein the express consent is received from the patient, or the authorized representative of the patient, via direct user input to the client computing device that provides access to the consent form; and
causing, by the medical consent application, updated consent resource data to be generated and stored at the medical provider computing device in response determining that the express consent for the medical procedure as received,
wherein the updated consent resource data includes an indication that the patient, or the authorized representative of the patient, consented to receiving the medical procedure.

9. The method of claim 8, wherein determining that the patient is to be presented with the consent form for consenting to the medical procedure includes:
determining that the medical records database includes medical procedure resource data,
wherein the medical procedure resource data includes another encrypted reference to the patient resource data and identifies the medical procedure.

10. The method of claim 9, wherein causing the consent resource data to be generated includes:
determining a location of an image file and/or a video file that provides supplemental information describing the medical procedure, and
causing the consent resource data to be generated with a link or an address for the image file and/or the video file,
wherein the image file and/or the video file are rendered at a graphical user interface (GUI) of the client computing device during or before the patient, or the authorized representative of the patient, providing the express consent at the consent form.

11. The method of claim 8, further comprising:
determining that the patient received the medical procedure subsequent to the express consent being provided at the consent form; and
causing, based on determining that the user received the portion of the medical procedure, discharge consent resource data to be generated,
wherein the discharge consent resource data identifies the medical procedure, and is accessible to the medical consent application, and includes another encrypted reference to the patient resource data stored at the medical records database.

12. The method of claim 8, further comprising:
determining that the patient received the medical procedure subsequent to express consent being provided at the consent form; and
causing, based on determining that the patient received the medical procedure, appointment resource data to be generated,
wherein the appointment resource data:
includes a reference to a file embodying post-procedure information for the medical procedure, and
specifies a time that a reminder will be communicated to a phone number and/or an email address of the patient or the authorized representative of the patient.

13. The method of claim 8, further comprising:
determining that the patient, or the authorized representative of the patient, provided an indication that a separate medical provider of the patient does not have an account stored at the medical provider computing device or is not associated with a network of the medical provider computing device; and
causing, by the medical consent application, the updated consent resource data to be converted to a separate format and communicated to an electronic address of the separate medical provider.

14. The method of claim 13, wherein the separate format of the updated consent resource data includes a consolidated clinical document architecture (CCDA) format.

15. A method implemented by one or more processors, the method comprising:
causing a medical consent application to generate patient resource data using provider patient data that is stored in a medical records database,
wherein the provider patient data is stored in a first format in the medical records database, and the patient resource data is generated in a second format;
determining that a patient is to undergo a medical process that can require consent from the patient, or an authorized representative of the patient, before the medical process is initiated by a provider,
wherein the patient is identified in the provider patient data that is stored in the medical records database, and the patient resource data includes an encrypted reference to the patient;
causing consent resource data to be generated in response to determining that the patient is scheduled to undergo the medical process that can require the consent,
wherein the consent resource data: is generated in the second format, and includes the encrypted reference to the patient or a different encrypted reference to the patient;
causing a patient consent form to be generated based on the consent resource data,
wherein the patient consent form includes natural language content data that:
describes the medical process, and is at least partially based on other data stored separately from the medical records database;
causing the natural language content data to be converted to audio data using a text-to-speech process,
wherein the natural language content data is converted by the medical consent application or a separate application; and
causing a graphical user interface (GUI) of a client computing device to render:
at least a portion of the patient consent form at an application interface of the medical consent application, and
a selectable element that, in response to being selected by a patient or a patient representative, causes playback of the audio data via an audio interface of the client computing device or a separate device.

16. The method of claim 15, further comprising:
causing the consent resource data to be updated to include a reference or link to the audio data subsequent to the natural language content data being converted to the audio data.

17. The method of claim 15, further comprising:
determining, subsequent to the GUI of the client computing device rendering the portion of the patient consent form, that playback of the audio data was performed at the client computing device or the separate device, and
causing the consent resource data to be updated to indicate a duration of the audio data that was played back.

18. The method of claim 15, further comprising:
generating updated consent resource data in response to the patient or the patient's providing an indication of consent to the patient consent form; and
causing the updated consent resource data to be communicated, in the first format, to the medical records database for storage,
wherein the updated consent resource data include a reference or link to an image that embodies the indication of consent to the patient consent form.

19. The method of claim 18, further comprising:
causing an updated consent form to be generated and communicated to an email address associated with the patient,
wherein the updated consent form is generated as a file or image that is attached to an electronic communication that embodies another reference or other link to the image that embodies the indication of the consent to the patient consent form.

20. The method of claim 18, wherein the electronic communication further embodies a particular reference or a particular link to the audio data for playback by a recipient of the electronic communication.
